SEQUENCE LISTING (4 sequences)

SEQ ID NO: 1 - DNA, 25 bp, Campylobacter jejuni
caccatggaaaaacaaaatattgcg

SEQ ID NO: 2 - DNA, 24 bp, Campylobacter jejuni
gctttccttgtgattaagaatgtt

SEQ ID NO: 3 - DNA, 37 bp, Neisseria meningitidis
gacgacgacaagatgcaaataaaaatagataaattaa

SEQ ID NO: 4 - DNA, 40 bp, Neisseria meningitidis
gaggagaagcccggttcattcaaaatcatcccatgttagt

(19) United States
(12) Patent Application Publication
(10) Pub. No.: US 2020/0332335 A1
(43) Pub. Date: Oct. 22, 2020

(54) MICROBIAL POLYSACCHARIDES AND METHODS OF USE

(71) Applicant: Morgan State University, Baltimore, MD (US)

(72) Inventors: Pumtiwitt McCarthy, Baltimore, MD (US); Sujan Ghimire, Baltimore, MD (US)

(21) Appl. No.: 16/842,923

(22) Filed: Apr. 8, 2020

Related U.S. Application Data
(60) Provisional application No. 62/831,319, filed on Apr. 9, 2019.

(51) Int. Cl. C12P 19/44; C07H 19/24; C12N 9/10; C12N 9/12

(52) U.S. Cl. CPC C12P 19/44; C07H 19/24; C12N 9/1241; C12Y 205/01057; C12Y 207/07043; C12N 9/1085

(57) ABSTRACT

Methods for removing heavy metals from contaminated water including contacting contaminated water with polysaccharides from N. meningitides serotypes B and W; a fusion gene product and fusion enzyme including silica acid synthase and CMP sialic acid synthetase, and use of the fusion enzyme in a simplified process to make CMP Sialic acid and derivatives thereof. Use of CMP Sialic acid and derivatives thereof to remove heavy metals from contaminated water.

### BACKGROUND OF THE INVENTION

### Field of the Invention

The present invention relates to microbial polysaccharides and methods for using them in water treatment.

### Description of the Background

Water pollution by heavy metals is an environmental and public health risk. Polluted water, usually caused by industrial waste byproducts, can negatively impact natural habitats of marine animals leading to disruption of ecosystems. Seafood or plants obtained from polluted sources for consumption and metal-polluted water is detrimental to human health. Metal poisoning can severely damage nearly all cellular components. Consequently, effective heavy metal removal from affected areas is extremely important. There has been an increase in the development of new heavy-metal capture methods that are eco-friendly. Bioremediation is a process that uses living organisms (mostly microorganisms and plants) rather than harsh chemicals to remove and/or detoxify waste products and pollutants. Microorganisms perform bioremediation either via biosorption or bioaccumulation. Biosorption is the removal of heavy metals by passive binding to non-living biomass in an aqueous solution. On the other hand, bioaccumulation is an active process which requires the metabolic activity of a living organism in the removal of metals.

Microbial exopolysaccharides (EPSs) can play key roles in biosorption. Bacteria, fungi and some algae are known to produce EPSs. EPSs can be found released into the environment or attached to the microorganism cell surface (in the case of capsular polysaccharides). Exopolysaccharides contain mostly polysaccharides, but can also contain nucleic acids, protein and phospholipids. Microbial EPSs play physiological roles in cell adhesion, biofilm formation and protection from host defense mechanisms. These biopolymers are equipped with ionizable functional groups that are known sites for interactions with heavy metal cations. These include groups such as carboxylic (—COOH), phosphoryl (—PO₄), amino (—NH₃) and hydroxyl (—OH) groups.

### SUMMARY OF THE INVENTION

Neisseria meningitidis serogroup W is one of six types of disease-causing serogroups of N. meningitidis. Accordingly, most studies with these polymers focus on vaccine development. The inventors, however, discovered that the polysaccharide structure binds strongly to heavy metal cations. Specifically, the inventors examined the metal-binding capacity of the capsular polysaccharides of N. meningitidis serogroup B and N. meningitidis serogroup W with Pb2+ and Cu2+ cations. The inventors discovered that serogroup B polysaccharide completely binds all metal concentrations and that serogroup W polysaccharide also binds at all metal concentrations, but somewhat less efficiently.

According to one embodiment of the invention, there is presented a method of using exopolysaccharides from Neisseria meningitides serogroups B and W to remove heavy metals from water. According to another embodiment of the invention, there is presented modified exopolysaccharides with improved heavy metal binding properties. Specially, according to various embodiments of the invention there is presented methods for optimizing the metal binding properties of an organism's capsular polysaccharide using the enzymatic machinery responsible for polysaccharide synthesis. Genetic engineering and recombinant DNA technology make it possible to design and optimize new biopolymers for this purpose. According to various alternative embodiments of the invention, there is presented modified polysaccharides from Neisseria meningitidis serogroup W with optimized binding properties. In addition, there is presented a fusion gene and fusion protein product for simplified production of the serogroup W polysaccharide (CMP-Sialic Acid) and modified versions thereof. The invention uses recombinant DNA technology to make a fusion protein of two enzymes needed for simplified biosynthesis of CMP-Sialic Acid. The invention further includes expressing the fusion protein in bacterial cells and use of the expressed fusion protein to synthesize CMP-Sialic Acid and modifications thereof for the removal of heavy metals from water.

Additional aspects of this invention include optimizing the binding properties of Neisseria meningitidis serogroup W polysaccharide using genetic engineering and recombinant DNA technology. According to this invention, modified and optimized serogroup W polysaccharide is enzymatically synthesized with improved metal-binding affinity for heavy metal capture, transformed into a plasmid vector for growth, expression, purification and characterization.

Accordingly, there is provided according to the invention a method of removing heavy metals from contaminated water comprising passing said contaminated water over an insoluble substrate to which is bound a compound selected from the group consisting of capsular polysaccharride of N. meningitidis serogroup B, capsular polysaccharide of N. meningitidis serogroup W, CMP-Sialic acid, derivatives thereof, and combinations thereof. According to preferred embodiments of the invention, the heavy metals are selected from the group consisting of cations of lead and copper and combinations thereof. According to further embodiments of the invention, the substrate may be inert and/or may be selected from the group consisting of water-insoluble organic and inorganic compounds and compositions. The substrate may comprise nanoparticles, nanotubes and/or polymeric resins. Suitable substrates include those inert and water-insoluble substrates used in the fields of bio-catalysis, bio-reactors and immobilization of enzymes, proteins and other biological materials and molecules.

According to further embodiments of the invention, there may be provided a DNA molecule comprising the DNA sequence of sialic acid synthase and the DNA sequence of CMP-Sialic acid synthetase.

According to another embodiment of the invention, there is provided a method of synthesizing CMP-Sialic acid comprising incubating, in a single reaction vessel without isolating intermediates, a fusion protein comprising the amino acid sequences of sialic acid synthase and CMP-Sialic acid synthetase with N-Acetylmannosamine, phosphoenolypyruvate and cytidine monophosphate. According to a related embodiment of the invention, there is provided a method of synthesizing derivatives of CMP-Sialic acid comprising incubating, in a single reaction vessel without isolating intermediates, a fusion protein comprising the amino acid sequences of sialic acid synthase and CMP-Sialic acid synthetase with analogs of N-Acetylmannosamine, phosphoenolypyruvate and cytidine monophosphate.

### BRIEF DESCRIPTION OF THE DRAWINGS

FIG. 1 shows the structure of the capsular polysaccharide of Neisseria meningitides B, where R represents acetylation sites.

FIG. 2 shows the structure of the capsular polysaccharide of Neisseria meningitides W, where R represents acetylation sites.

FIG. 3 is a chart showing lead concentrations in filtrate (red) and retentate (blue) in control samples for lead-binding experiments.

FIG. 4 is a chart showing lead concentrations in filtrate (black) and retentate (red) in reaction samples containing capsular polysaccharide of Neisseria meningitides B.

FIG. 5 is a chart showing copper concentrations in filtrate (red) and retentate (blue) in control samples for copper-binding experiments.

FIG. 6 is a chart showing copper concentrations in filtrate (black) and retentate (red) in reaction samples containing capsular polysaccharide of Neisseria meningitides B.

FIG. 7 is a chart showing lead concentrations in filtrate (red) and retentate (blue) in control samples for lead-binding at 50 mg/L.

FIG. 8 is a chart showing lead concentrations in filtrate (red) and retentate (blue) in reaction samples containing capsular polysaccharide of Neisseria meningitides W.

FIG. 9 shows the structure of CMP-Sialic Acid, including various substitutions according to alternative embodiments of the invention.

### DETAILED DESCRIPTION

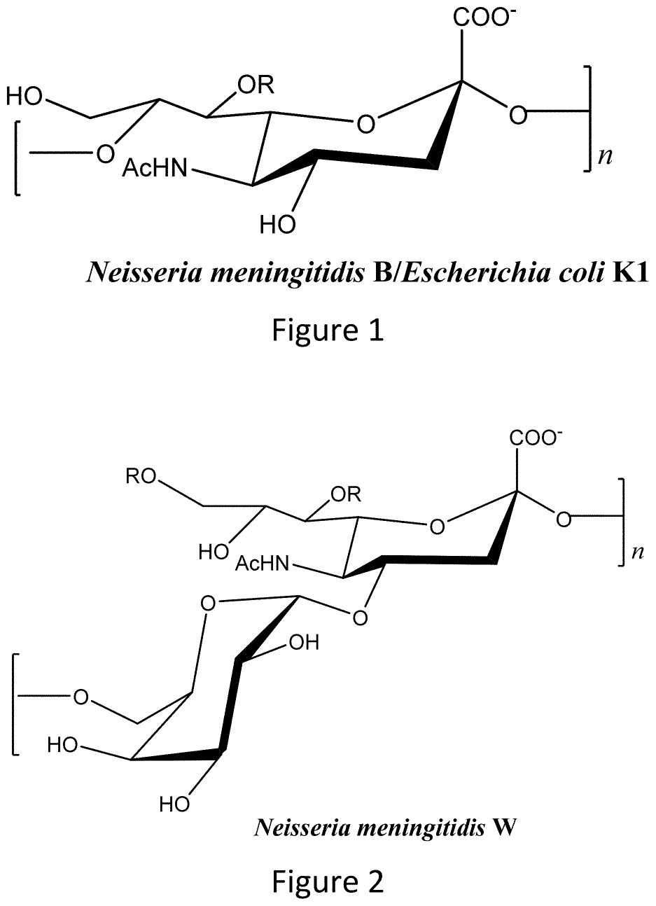

Neisseria meningitidis serogroup B capsular polysaccharide (FIG. 1) is a homopolymer of α-2,8-linked N-acetylneuraminic acid. N. meningitidis serogroup W capsular polysaccharide (FIG. 2) is a heteropolymer of repeating units of an α-1,4 linked galactose-sialic acid, CMP-Sialic acid (cytidine-5′monophospho-N-acetylneuraminic acid).

For the determination of metal binding to N. meningitidis capsular polysaccharides, 1 mg/mL of polysaccharide is made by dissolving 0.01 g of polysaccharide in 10 mL of ultrapure, distilled water. A stock concentration of lead (250 mg/L) is made by dissolving 0.0025 g of Lead (II) nitrate or Copper (II) nitrate in 10 mL of ultrapure, distilled water. Six different working concentrations (5 mL each) of lead (5 mg/L, 10 mg/L, 20 mg/L, 30 mg/L, 40 mg/L and 50 mg/L) are made by appropriate dilutions of the stock solution. Each sample is incubated with either 1 mL of ultrapure filtered water (controls) or with 1 mL of polysaccharide. The experiment is performed in duplicate. Both controls and reactions are shaken at 200 RPM for 2 hours at room temperature. After 2 hours, a total of 3 mL of sample is passed through an Ultracel-3 membrane, 3 kDa cutoff via centrifugation for 20 minutes at 6000 RPM. After centrifugation the metal concentration in both filtrate, supernatant and unfiltered samples are analyzed using an atomic absorption spectrometer. The same method is used for both Neisseria meningitidis serogroup B and serogroup W polysaccharides.

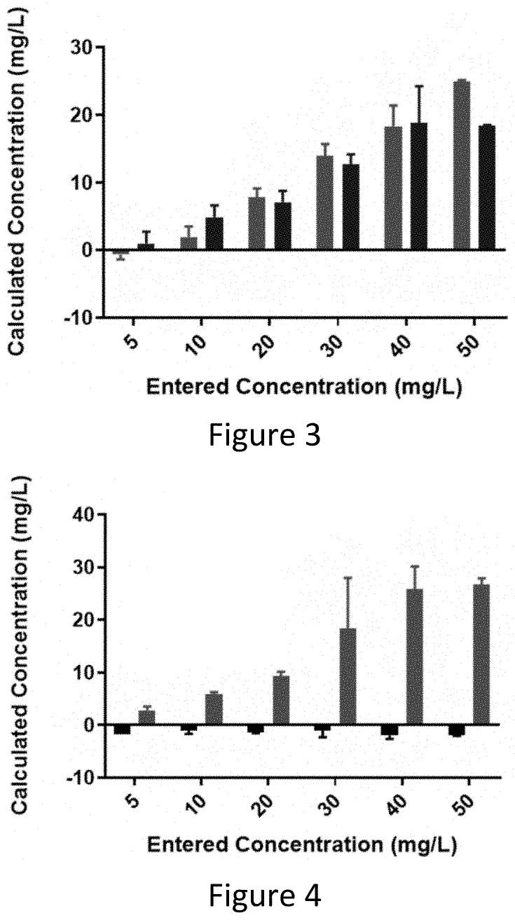

Metal-binding is assessed after 2 hr incubation for control and reaction samples. After this time, 50% of these reactions (3 mL of 6 mL total) are passed through a 3 kDa cutoff filtration device. Polysaccharides and anything complexed to the polysaccharide will remain in the retentate and any free metal will pass through the filter. The free metal concentration is determined for both unfiltered and filtered control and reaction samples. In testing of Pb2+ metal binding, the same initial concentration of metal is found to be present in both unfiltered control and reaction samples. This indicates that the initial metal concentration is the same for both conditions. For filtered control samples, equal concentrations of metals are found to be present in both the filtrate and supernatant indicating that unbound metals are freely able to pass through the filter (FIG. 3). In the case of reaction samples after filtration (FIG. 4) no metal is found to be present in filtrate indicating formation of a polysaccharide-metal complex. This complex is not able to pass through the filter. All metal is polysaccharide-bound because the only metal present is found in the supernatant. The metal content of polysaccharide is also tested and no metal is present, which indicates that any metal found in the supernatant is there because it is bound to polysaccharide.

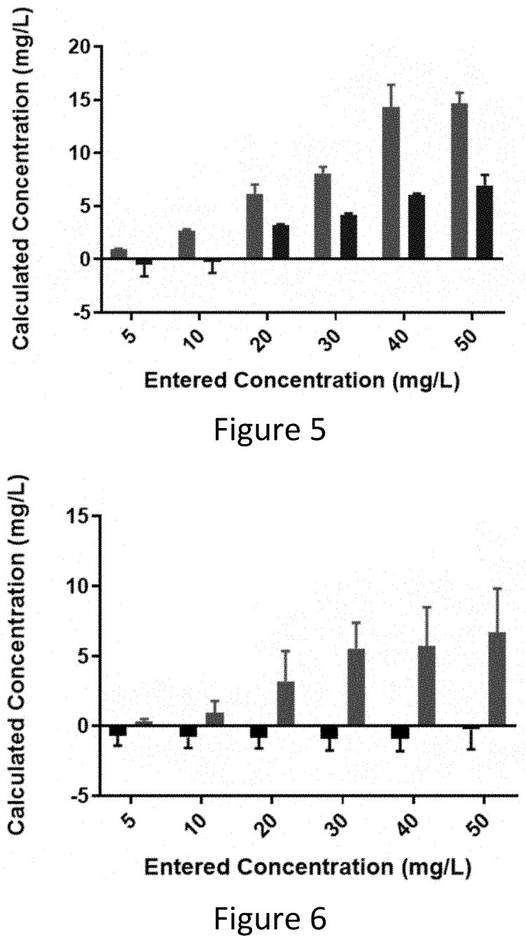

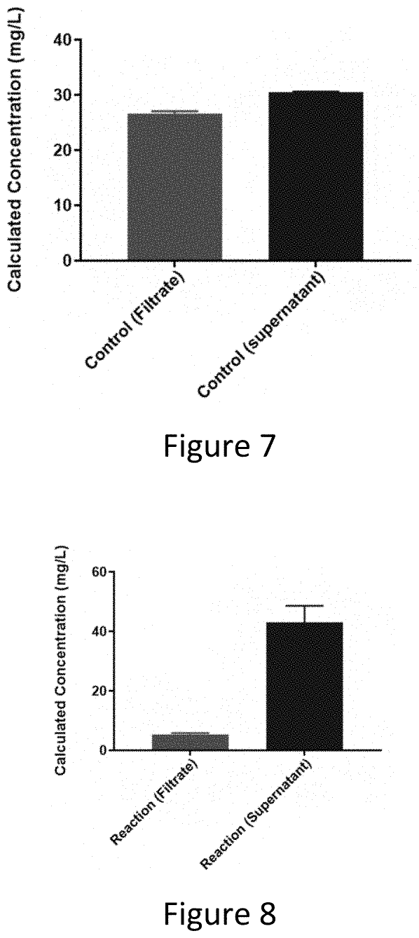

The observed results for copper are like those seen for lead however there is less free metal in the supernatant compared to filtrate in the filtered control samples (FIG. 5). No metal is found to be present in filtrate indicating formation of a complex (FIG. 6). All metal is present only in the supernatant.

Where Neisseria meningitidis serogroup B capsular polysaccharide contains only repeating units of negatively charged sialic acid, the polysaccharide of serogroup W contains repeating unit of both neutral sugar galactose and negatively charged sialic acid. The same trends are found with lead binding to this polysaccharide as is found for serogroup B capsular polysaccharide. The same initial concentration of metal is found to be present in both unfiltered control and reaction samples. In filtered control samples equal concentrations of Pb2+ ion is found to be present in both filtrate and supernatant (FIG. 7). In reaction samples, some Pb2+ cations (approx. 5 mg/L) is found to be present in filtrate and approximately 40 mg/mL is found in the supernatant (FIG. 8). This might be due to the difference in composition of two polysaccharides. The serogroup W polysaccharide has fewer negatively charged functional groups to bind the cations which may explain why some unbound metal appeared in the filtrate (compare FIGS. 1 and 2).

Sialic acid synthase (SAS) catalyzes formation of sialic acid through a condensation reaction between the sugar N-acetylmannosamine (ManNAc) and phosphoenolypyruvate. CMP-Sialic acid synthetase (CSS) attaches a cytidine monophosphate to a sialic acid residue. When these reactions are coupled together, SAS produces sialic acid and CSS attaches a cytidine monophosphate molecule to that sialic acid to yield a CMP-Sialic acid molecule as shown below:

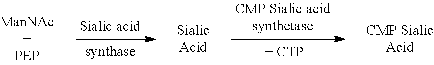

According to a further embodiment of the invention, there is provided a recombinant gene fusion product of sialic acid synthase (SAS) produced by Campylobacter jejuni bacterium and CMP-Sialic acid synthetase (CSS) produced by Neisseria meningitidis bacterium for expression of a fusion enzyme for the simplified (single batch) synthesis of CMP-Sialic acid for environmental treatment of heavy metals.

Overnight cultures of C. jejuni SAS and Neisseria meningitidis CSS (both expressed in non-toxic E. coli KRX cells) are used to extract the plasmid DNA for further studies. The plasmid DNA is purified using Zyppy™ Plasmid Miniprep kit. Quantitation of DNA followed purification using NanoDrop instrument. The SAS and CSS sequences are isolated out of the plasmid and amplified using sequence specific primers shown in Table 1. Purification and analysis of PCR products are performed via agarose gel electrophoresis.

TABLE 1
Primers used to isolate and amplify CSS
and SAS genes

| Template | | |
|---|---|---|
| DNA | Forward Primer | Reverse Primer |
| CSS | CACCATGGAAAAACAA | GCTTTCCTTGTGATTA |
| | AATATTGCG | AGAATGTT |
| SAS | GACGACGACAAGATGC | GAGGAGAAGCCCGGTT |
| | AAATAAAAATAGATAA | CATTCAAAATCATCCC |
| | ATTAA | ATGTTAGT |

To create the fusion gene, primers are designed to amplify fragments with appropriate overlaps. The SAS DNA fragment is amplified with primers containing the overlap region of CSS DNA sequence. The CSS DNA fragment is amplified with primers containing the overlap region of SAS DNA sequence. Primers are created using the online NEBuilder Assembly Tool.

DNA fragments containing overlaps are fused together using NEBuilder® HiFi DNA Assembly Master Mix to create fusion enzyme gene products (fusing SAS & CSS together). Fused fragments contain each order: CSS first, then SAS, and SAS first, then CSS.

The SAS-CSS and CSS-SAS fragments are spliced into an expression vector, which vector is introduced into a bacterial host cell. The protein synthesis mechanism of the host cell produces the SAS-CSS or CSS-SAS fusion protein encoded by the fusion genes, and the expressed fusion protein is harvested and purified. The purified SAS-CSS fusion protein is used in a single batch reaction to catalyze formation of sialic acid via condensation reaction between ManNAc and phosphoenolypyruvate (both available from Sigma-Adrich), followed by attachment of a cytidine monophosphate from cytidine triphosphate (also available from Sigma-Aldrich) to the sialic acid to produce CMP-Sialic acid.

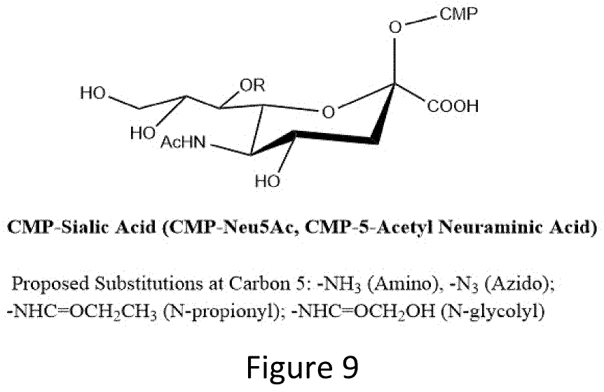

To produce modified CMP-Sialic acid with enhanced metal binding affinities, selected analogs of the natural sialic acid precursor sugar N-Acetylmannosamine (ManNAc) and or the cytidine monophosphate are used to replace ManNAc in the sialic acid biosynthesis pathway resulting in production of a corresponding sialic acid derivative according to Table 2 and FIG. 9. Mannosamine derivatives will help determine whether the acetylation of the amino group is key to the metal binding function within the sugar. Additional substitutions include longer alkyl chain length (N-acetyl vs. N-propionyl); removal of oxygen (azido) and adding a hydroxyl group (glycoyl).

TABLE 2
Synthesis of CMP-Sialic Acid Derivatives

| | CMP-Sialic Acid (CMP- |
|---|---|
| ManNAc/Analog | Neuraminic Acid) Derivative |
| Mannosamine | CMP-Aminoneuraminic Acid |
| N-propanoylmannosamine | CMP-N-Propanoylneuraminic Acid |
| Azidomannose | CMP-Azidoneuraminic Acid |
| N-Glycolylmannosamine | CMP-Glycolylneuraminic Acid |

CMP-Sialic Acid derivatives so-produced are examined for metal binding affinity using the methods described herein above, and using surface plasmon resonance. Additionally, computer modeling of derivatives may be employed to optimize selection of precursors. Claims:

1. A method of removing heavy metals from contaminated water comprising passing said contaminated water over an inert water-insoluble substrate to which is bound a compound selected from the group consisting of capsular polysaccharide of N. meningitidis serogroup B, capsular polysaccharide of N. meningitidis serogroup W, CMP-Sialic acid, derivatives thereof, and combinations thereof.

2. The method of claim 1, wherein the heavy metals are selected from the group consisting of cations of lead and copper and combinations thereof.

3. A DNA molecule comprising the DNA sequence of sialic acid synthase and the DNA sequence of CMP-Sialic acid synthetase.

4. A method of synthesizing CMP-Sialic acid comprising incubating, in a single reaction vessel without isolating intermediates, a fusion protein comprising the amino acid sequences of sialic acid synthase and CMP-Sialic acid synthetase with N-Acetylmannosamine, phosphoenolypyruvate and cytidine triphosphate.

5. A method of synthesizing derivatives of CMP-Sialic acid comprising incubating, in a single reaction vessel without isolating intermediates, a fusion protein comprising the amino acid sequences of sialic acid synthase and CMP-Sialic acid synthetase with analogs of N-Acetylmannosamine, phosphoenolypyruvate and cytidine triphosphate.
